(19) United States
(12) Patent Application Publication
(10) Pub. No.: US 2025/0387637 A1
(43) Pub. Date: Dec. 25, 2025

(54) HELMET-TYPE MAGNETIC STIMULATION DEVICE

(71) Applicant: REMED BRAINSTIM CO., LTD., Seongnam-si (KR)

(72) Inventors: Se Jin YOON, Anyang-si (KR); Il Kyu KIM, Suwon-si (KR); Sung Taek CHO, Seoul (KR); Young Kwang KIM, Seoul (KR)

(21) Appl. No.: 18/728,549

(22) PCT Filed: Jan. 17, 2023
(86) PCT No.: PCT/KR2023/000784

§ 371 Date: Apr. 17, 2025

(30) Foreign Application Priority Data
Jan. 25, 2022 (KR) 10-2022-0010898
May 23, 2022 (KR) 10-2022-0062600

(51) Int. Cl. A61N 2/00; A61N 2/02

(52) U.S. Cl. CPC A61N 2/006; A61N 2/02

(57) ABSTRACT

The present invention relates to a helmet-type magnetic stimulation device for providing non-invasive treatment by applying a magnetic field to cranial nerve cells or scalp cells, the helmet-type magnetic stimulation device comprising: a helmet-type case part; a magnetic field generation part for generating a magnetic field in order to apply a magnetic field to the head; and a fixing part installed on one inner side of the case part to fix and support the magnetic field generation part at a predetermined position on the head. Therefore, the present invention includes a magnetic field generation part for generating a magnetic field in order to apply a magnetic field to the head, inside the case part.

### TECHNICAL FIELD

The present invention disclosed herein relates to a helmet-type magnetic stimulation device, and more particularly, to a helmet-type magnetic stimulation device for applying a magnetic field to a scalp or a cranium of a human body to perform non-invasive treatment.

### BACKGROUND ART

Transcranial Magnetic Stimulation (hereinafter referred to as “TMS”) is brain stimulation which uses local magnetic pulses induced on the outer surface of the head of the body to activate or inhibit nerve cells in specific areas of the brain. The principle of TMS is that an intensive electric current is applied to an electromagnetic coil to generate a magnetic field having an intensity of about several Tesla. This is a method of stimulating the brain by delivering fluctuation energy of these magnetic pulses to the brain to induce depolarization in nerve cells to a range of about several centimeters (cm) beneath the coil.

TMS is a representative non-invasive brain stimulation, and therapeutic applications for depression, insomnia, obsession, dyskinesia, dementia, brain dysfunction, and the like, which do not well react to medication and psychotherapy, are actively researched.

Generally, a suggested TMS session is carried out once a day for about 20 minutes, five days a week, and intensive treatment is required. For example, in the case in which the magnetic stimulation treatment held for about 20-50 minutes per day was repeatedly applied, for several days, for central nervous diseases such as stroke or spinal cord injury, neuroplasticity or neural regeneration and functional recovery effects are reported.

However, transcranial magnetic stimulation devices according to the related art have shapes impossible to move, and thus have difficulty to meet this treatment cycle under domestic medical market conditions. Thus, there is a need to introduce reduced-size TMS devices that are conveniently available for home use.

Meanwhile, hair loss refers to a lack of hair on parts of the scalp which should have been normally covered with hair. This hair loss is known to occur due to various causes such as genetics, action of male hormones, stress, and environmental pollution. Recently, as patients with hair loss who are young people aged 20 to 30 years and women increase, the hair loss is emerging as a serious social issue, and also interests about hair loss treatment devices are on an increasing trend.

In recent years, a helmet-type treatment device, which includes a treatment machine having protrusions installed with laser diodes or the like and generates a wavelength optimal for growth of hair, attracts the most interest as a hair loss treatment device for home use. This helmet-type treatment device activates various cell tissues including hair roots by emitting laser light onto the scalp so that optical energy penetrates dermis tissues and subcutaneous tissues.

However, there is a problem that this technique according to the related art depends only on the light therapy simply using laser, and thus is not very effective in treatment. And it is sought to promote hair growth and prevent hair loss by adding vibration therapy or ultrasound therapy to activate biological tissues of the scalp through a negative pressure, but the method of activating the tissues of the scalp through combination with the vibration or ultrasound has also a problem that complex action of light and vibration or ultrasound is not significant, and thus effects thereof are not high.

### DISCLOSURE OF THE INVENTION

### Technical Problem

The present invention has been devised to solve the problems as above, and an object of the present invention is to provide a helmet-type magnetic stimulation device including a magnetic field generation part, which generates a magnetic field so as to apply a magnetic field to a scalp or cranium, and a cooling part which cools heat generated from the magnetic field generation part.

The present invention also provides a helmet-type magnetic stimulation device in which a lower case part and an upper case part, as the case part, are coupled to be attached/detached to/from each other, and a lower case is provided with a spacing piece to maintain a spaced distance from the scalp to be constant so that the magnetic field may be easily applied and also a applied range of the magnetic field may be increased, and installation for magnetic field generation may be easily achieved.

The present invention also provides a helmet-type magnetic stimulation device in which the magnetic field generation part is provided as a wound coil so that an intensity or a shape of the magnetic field may be adjusted according to treatment progression in a user, and blood flow in a scalp tissue may be promoted to be enhanced.

The present invention also provides a helmet-type magnetic stimulation device in which a cooling fan and a cooling hole, as the cooling part, are disposed in various positions of the casing part so that outside air may be easily circulated, and also heat generation of the magnetic field generation part may be efficiently controlled to optimize cooling performance for cooling heat generated by the magnetic field.

### Technical Solution

The present invention for achieving the purposes as above is a helmet-type magnetic stimulation device for providing non-invasive treatment by applying a magnetic field to cranial nerve cells or scalp cells, the helmet-type magnetic stimulation device including: a helmet-type case part; a magnetic field generation part (30) which is installed in an inner space of the case part and generates the magnetic field; and a fixing part (50) which is installed inside the case part in one direction, and fixes and supports the magnetic field generation part (30) at a predetermined position above a cranium.

The case part according to the present invention includes: a lower case part (10) mounted to be spaced a predetermined distance upward from the cranium; and an upper case part (20) coupled to an upper portion of the lower case part (10) to define an inner space.

The lower case part (10) according to the present invention includes: a lower case mounted to be spaced a predetermined distance upward from the cranium; a cooling guide provided upright on an upper surface of the lower case in a longitudinal direction; a coupling piece which protrudes from an outer circumference of the lower case and to which the upper case part (20) is coupled; and a spacing piece which is provided in plurality to protrude from a lower surface of the lower case and is brought into contact with a scalp surface.

The magnetic field generation part (30) according to the present invention includes wound coils in a plurality of layers spaced a predetermined winding distance from each other. The present invention further includes a spacer (40) which supports lower portions of the wound coils so as to maintain the winding distance of the wound coils to be constant.

The spacer (40) according to the present invention includes: a spacing plate installed in the inner space of the case part; a first spacing groove recessed in each of both ends of an upper surface of the spacing plate in one direction; a second spacing groove recessed in each of both ends of the upper surface of the spacing plate in the other direction; and a support piece extending outward from an outer circumference of the spacing plate.

The fixing part (50) according to the present invention includes at least one of: a first fixing piece which is installed on a central area of the case part, and fixes and supports the magnetic field generation part (30) at a central area of the cranium; a second fixing piece which is installed on a front area of the case part, and fixes and supports the magnetic field generation part (30) at a front area of the cranium; or a third fixing piece which is installed on a rear area of the case part, and fixes and supports the magnetic field generation part (30) at a rear area of the cranium.

### ADVANTAGEOUS EFFECTS

As described above, the present invention provides the helmet-type magnetic stimulation device including the magnetic field generation part which generates the magnetic field so as to apply the magnetic field to the scalp or cranium, and the cooling part which cools the heat generated from the magnetic field generation part. Accordingly, the user may carry out the scalp or cranium stimulation without the time and space constraints.

Also, according to the helmet-type magnetic stimulation device according to the present invention, the specified brain nerve cells are stimulated at the position on which the magnetic field generation part is disposed, thereby enabling the non-invasive treatment of depression, insomnia, obsession, dyskinesia, and brain dysfunction such as dementia.

Also, according to the helmet-type magnetic stimulation device according to the present invention, the magnetic field generation part which generates the magnetic field so as to apply the magnetic field to the scalp is included to smoothly circulate the blood in the scalp and stimulate the scalp and the hair roots. Accordingly, the hair loss may be prevented, and the hair loss may be treated.

Also, the lower case part and the upper case part, as the case part, are coupled to be attached/detached to/from each other, and the lower case is provided with the spacing piece, thereby providing the effects that the spaced distance from the scalp may be maintain to be constant to easily apply the magnetic field and also increase the applied range of the magnetic field, and to easily achieve the installation for the magnetic field generation.

Also, the wound coils in the plurality of layers are provided as the magnetic field generation part, and are supported by the spacing support part so that the winding distance is uniform, thereby providing the effects that the generation of the magnetic field may be uniformly applied to the cranium or the scalp to maintain the efficacy of the magnetic field uniformly.

Also, the case part is provided in the helmet-type, thereby providing the effects that the wearing may be facilitated and also the cranium stimulation or the hair loss treatment may be combined, and the user-specific treatment may be carried out while adjusting the applied intensity of the magnetic field and the duration of use.

Also, the magnetic field generation part is utilized as the heat dissipating body and cooled variously, thereby providing the effects that the assembly structure and size as a whole and the arrangement configuration may be simplified, and the manufacturing costs of the helmet-type magnetic stimulation device may be reduced.

Also, the wound coils may be manufactured in various sizes, and the arrangement of the wound coils may be optimized to enable the wound coils to be arranged in the array form at the proper position, thereby providing the effect that the magnetic stimulation efficiency may be maximized.

Also, the cooling fan and the cooling hole, as the cooling part, are disposed in the various positions of the casing part, providing the effects that the outside air may be easily circulated and also the heat generation of the magnetic field generation part may be efficiently controlled to optimize the cooling performance for cooling the heat generated by the magnetic field.

### BRIEF DESCRIPTION OF THE DRAWINGS

FIG. 1 is a schematic view illustrating a helmet-type magnetic stimulation device according to an embodiment of the present invention.

FIG. 2 is an exploded view illustrating a case part of a helmet-type magnetic stimulation device according to an embodiment of the present invention.

FIG. 3 is an exploded perspective view illustrating a helmet-type magnetic stimulation device according to an embodiment of the present invention.

FIG. 4 is a detailed view illustrating a fixing part of a helmet-type magnetic stimulation device according to an embodiment of the present invention.

FIG. 5 is a view illustrating a used state of a helmet-type magnetic stimulation device according to an embodiment of the present invention.

FIG. 6 is a view illustrating a first installed state of a magnetic field generation part of a helmet-type magnetic stimulation device according to an embodiment of the present invention.

FIG. 7 is a plan view illustrating a first installed state of a magnetic field generation part of a helmet-type magnetic stimulation device according to an embodiment of the present invention.

FIG. 8 is a view illustrating a second installed state of a magnetic field generation part of a helmet-type magnetic stimulation device according to an embodiment of the present invention.

FIG. 9 is a plan view illustrating a second installed state of a magnetic field generation part of a helmet-type magnetic stimulation device according to an embodiment of the present invention.

FIG. 10 is a view illustrating a third installed state of a magnetic field generation part of a helmet-type magnetic stimulation device according to an embodiment of the present invention.

FIG. 11 is a plan view illustrating a third installed state of a magnetic field generation part of a helmet-type magnetic stimulation device according to an embodiment of the present invention.

FIG. 12 is a cross-sectional view illustrating a helmet-type magnetic stimulation device according to an embodiment of the present invention.

### MODE FOR CARRYING OUT THE INVENTION

Hereinafter, preferred embodiments of the present invention will be described in more detail with reference to the accompanying drawings.

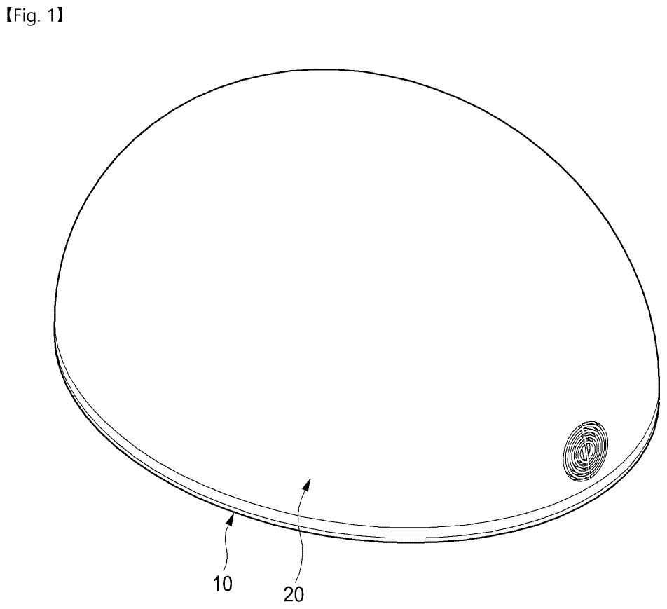

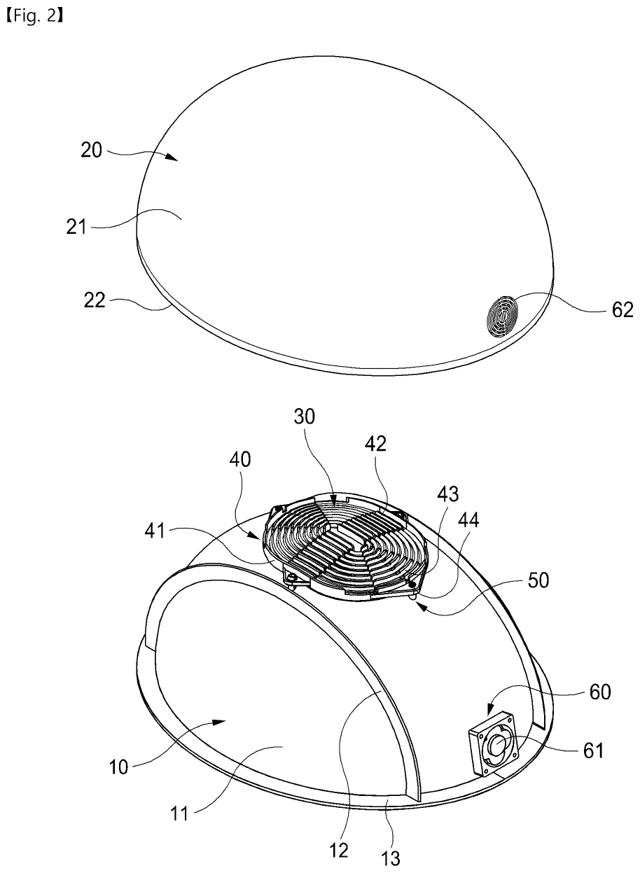

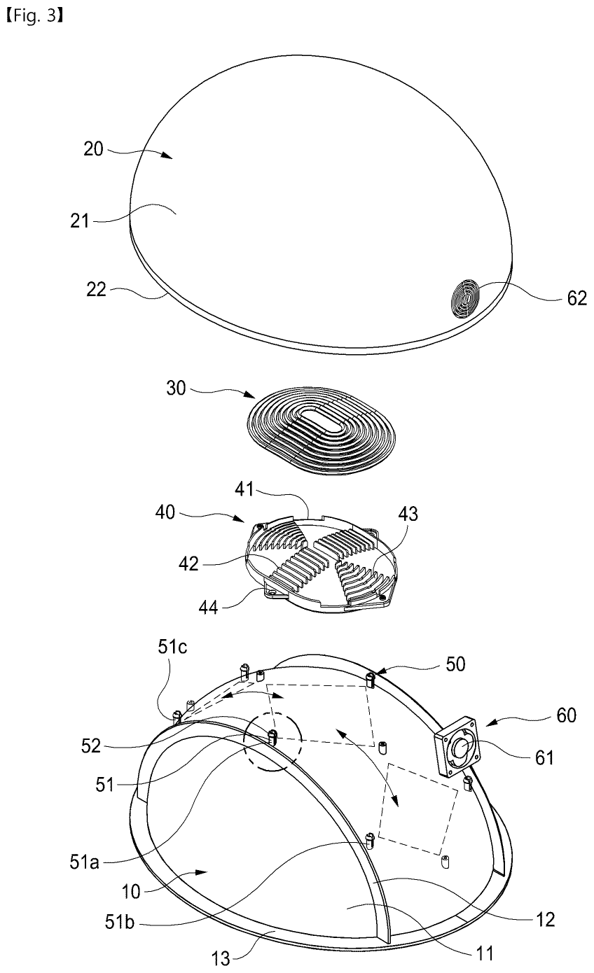

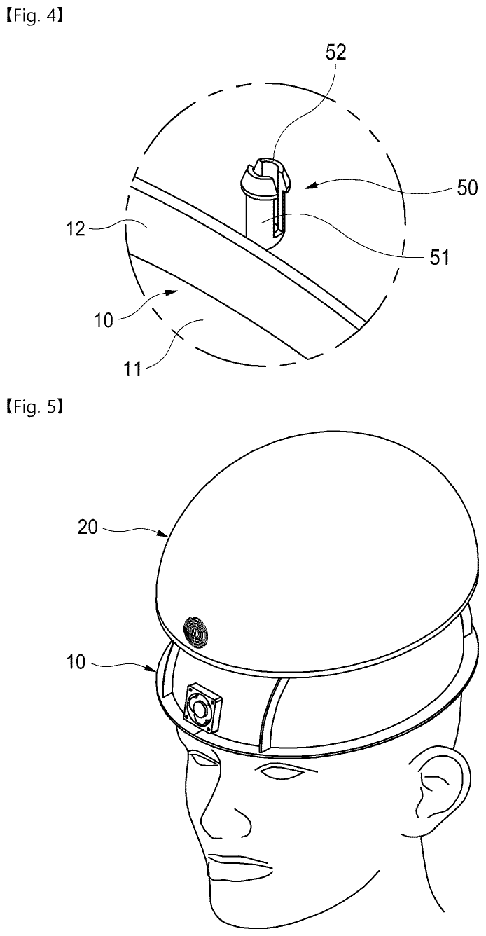

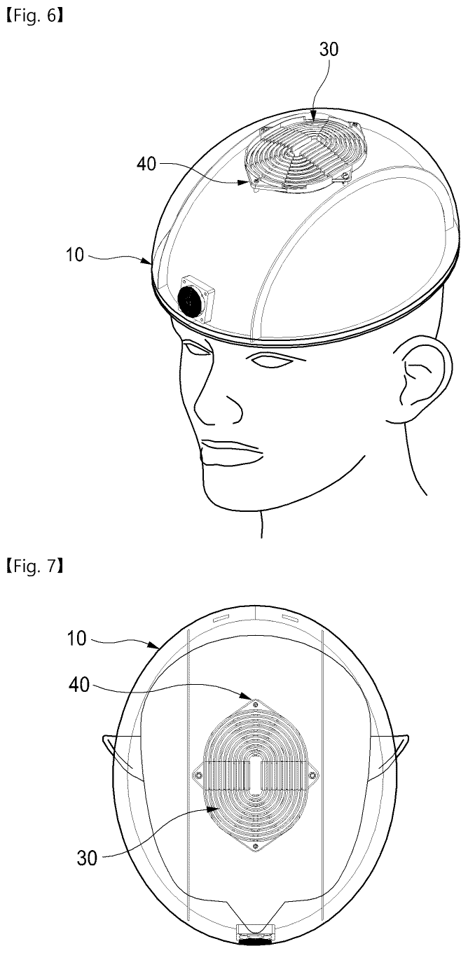

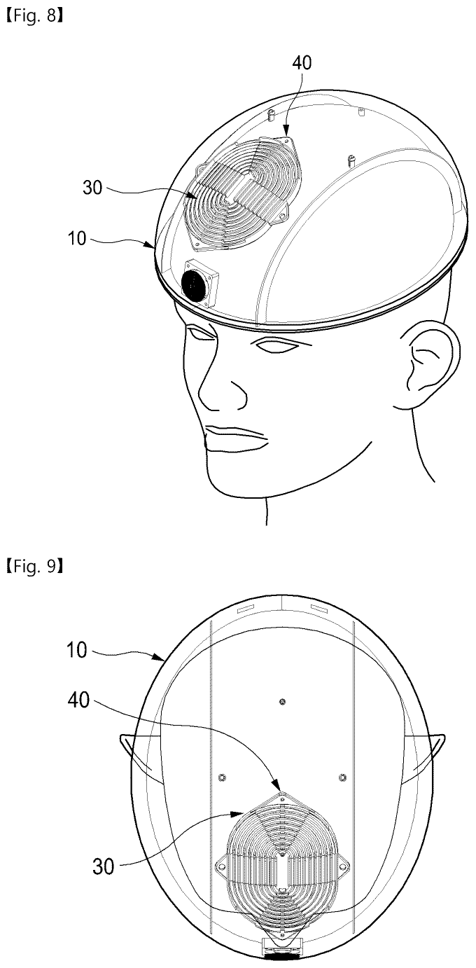

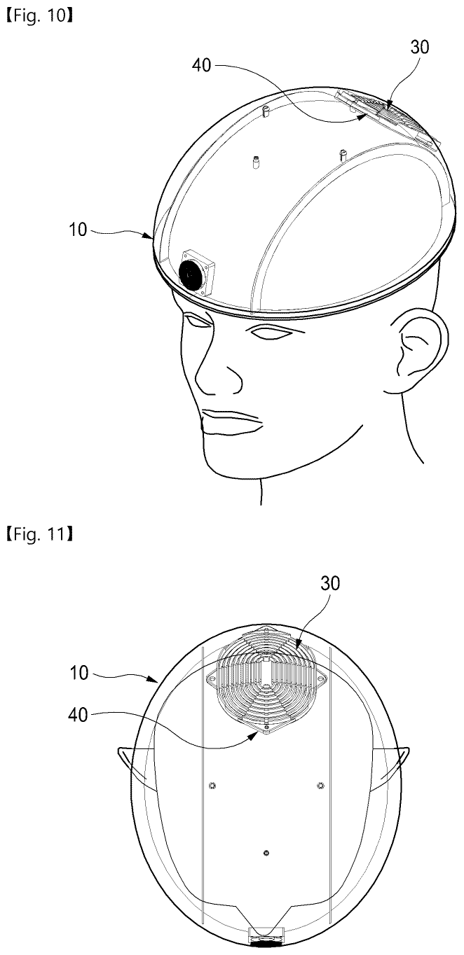

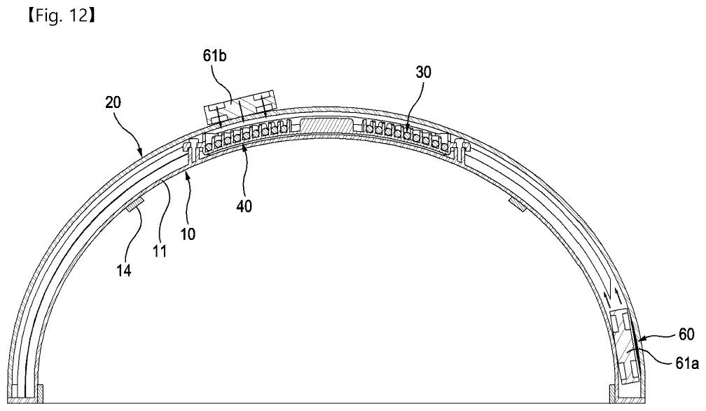

FIG. 1 is a schematic view illustrating a helmet-type magnetic stimulation device according to an embodiment of the present invention. FIG. 2 is an exploded view illustrating a case part of a helmet-type magnetic stimulation device according to an embodiment of the present invention. FIG. 3 is an exploded perspective view illustrating a helmet-type magnetic stimulation device according to an embodiment of the present invention. FIG. 4 is a detailed view illustrating a fixing part of a helmet-type magnetic stimulation device according to an embodiment of the present invention. FIG. 5 is a view illustrating a used state of a helmet-type magnetic stimulation device according to an embodiment of the present invention. FIG. 6 is a view illustrating a first installed state of a magnetic field generation part of a helmet-type magnetic stimulation device according to an embodiment of the present invention. FIG. 7 is a plan view illustrating a first installed state of a magnetic field generation part of a helmet-type magnetic stimulation device according to an embodiment of the present invention. FIG. 8 is a view illustrating a second installed state of a magnetic field generation part of a helmet-type magnetic stimulation device according to an embodiment of the present invention. FIG. 9 is a plan view illustrating a second installed state of a magnetic field generation part of a helmet-type magnetic stimulation device according to an embodiment of the present invention. FIG. 10 is a view illustrating a third installed state of a magnetic field generation part of a helmet-type magnetic stimulation device according to an embodiment of the present invention. FIG. 11 is a plan view illustrating a third installed state of a magnetic field generation part of a helmet-type magnetic stimulation device according to an embodiment of the present invention. FIG. 12 is a cross-sectional view illustrating a helmet-type magnetic stimulation device according to an embodiment of the present invention.

As illustrated in FIGS. 1 to 3, a helmet-type magnetic stimulation device according to this embodiment is a helmet-type magnetic stimulation device which includes a case part, a magnetic field generation part 30, a spacer 40, a fixing part 50, and a cooling part 60, and performs transcranial magnetic stimulation or applies a magnetic field to a scalp to treat hair loss.

The case part is a case member mounted above a cranium of the human body, and includes a lower case part 10 mounted to be spaced a predetermined distance upward from the cranium of the human body, and an upper case part 20 coupled to an upper portion of the lower case part 10 to define an inner space.

The lower case part 10 is a case member mounted to be spaced a predetermined distance upward from the cranium of the human body, and includes a lower case 11, a cooling guide 12, a coupling piece 13, and a spacing piece 14.

The lower case 11 is a case member mounted to be spaced a predetermined distance upward from the cranium of the human body, and includes a case that is convex upward to be brought into contact with the cranium of the human body, and mounted in a state of being spaced a predetermined distance from the scalp of the human body.

The cooling guide 12 is a guide member provided upright upper surface of the lower case 11 in a longitudinal direction, and the cooling guide 12 is provided upright on both ends of the upper surface of the lower case 11 in the longitudinal direction that is a front and rear direction, and guides outside air introduced by the cooling part 60 to the magnetic field generation part 30.

The coupling piece 13 is a coupling member, which protrudes from an outer circumference of the lower case 11 and to which the upper case part 20 is coupled, and the coupling piece 13 is provided as a hooking member protruding along the outer circumference of the lower case 11, and is hooked and coupled to the upper case part 20.

The spacing piece 14 is a spacing member which is provided in plurality to protrude from a lower surface of the lower case 11 and is brought into contact with the cranium of the human body, and the spacing piece 14 is brought into contact with the cranium of the human body and maintains a spaced distance from the lower surface of the lower case 11 to be constant. Accordingly, a spaced distance between the magnetic field generation part 30 and the cranium of the human body may be also maintained to be constant.

The upper case part 20, as illustrated in FIGS. 3 and 4, is a case member, which is detachably coupled to the upper portion of the lower case part 10 to define the inner surface between the upper case part 20 and the lower case part 10, and includes an upper case 21 and a connecting piece 22.

The upper case 21 is a case member detachably hooked and coupled to an upper portion of the lower case 11, and defines a cooling space and the inner space in which the magnetic field generation part 30 is installed between the lower case part 10 and the upper case part 20.

The connecting piece 22 is a connecting member which is recessed in an outer circumference of the upper case 21 and to which the upper case part 20 is coupled, and the connecting piece 22 is provided as a hooking groove recessed along an outer circumference of the upper case 21 and is hooked and coupled to the coupling piece 13 of the lower case part 10.

The magnetic field generation part 30 is a magnetic field generation member that is installed in the inner space of the case part and generates a magnetic field so as to apply the magnetic field to the cranium, and the magnetic field generation part 30 includes wound coils in a plurality of layers spaced a predetermined winding distance from each other or includes a transducer coil having a cylindrical annular shape or disc shape, and is disposed in the case part to generate the magnetic field.

In addition, a microscopic magnetic field having an electric field of 1 Hz to 50 Hz and 1 V/m to 150 V/m, which is a magnetic field generation range of the magnetic field generation part 30, reaches only the scalp and the subcutaneous layer without reaching a cranial bone of the human body. Thus, the microscopic magnetic field is suitable for the hair loss treatment.

For other treatment of various indicants such as depression, autism, dementia, and insomnia, the magnetic field generation range of the magnetic field generation part 30 may be adjusted.

This wound coil is a coil member, which is installed in the inner space of the case part and generates magnetism, and generates a pulsed magnetic field through a current applied thereto. As illustrated in FIGS. 2 and 3, this wound coil is separated by interposing the spacer 40 installed on the upper surface of the lower case part 10, and includes a coil winding layer in which the coils are wound in the plurality of layers having an circular shape or an oval shape.

The spacer 40 is a spacing member, which supports a lower portion of the wound coil so that the winding distance of the wound coil is maintained to be constant, and includes a spacing plate 41, a first spacing groove 42, a second spacing groove 43, and a support piece 44.

The spacing plate 41 is a plate member, which is installed in the inner space between the lower case part 10 and the upper case part 20, and is fixed and installed on the upper surface of the lower case part 10 with the fixing part 50 interposed therebetween.

The first spacing groove 42 is a spacer member in which a plurality of spacing grooves equally spaced are recessed in both ends of the upper surface of the spacing plate 41 in one direction, and both ends of the wound coil of the magnetic field generation part 30 in the one direction are fitted and coupled to the plurality of spacing grooves, respectively, so that the winding distance of the wound coils is maintained to be constant.

The second spacing groove 43 is a spacer member in which a plurality of spacing grooves equally spaced are recessed in both ends of the upper surface of the spacing plate 41 in the other direction, and both ends of the wound coil of the magnetic field generation part 30 in the other direction are fitted and coupled to the plurality of spacing grooves, respectively, so that the winding distance of the wound coils is maintained to be constant.

The support piece 44 is a support member extending outward from an outer circumference of the spacing plate 41. The support piece 44 extends from each of outer areas in four directions that are front, rear, left, and right directions of the spacing plate 41, and the fixing part 50 is fitted and coupled thereto so that the spacing plate 41 is fixed and supported by the lower case 10.

The fixing part 50 is a fixing member, which is installed inside the case part in one direction to fix and support the magnetic field generation part 30 at a predetermined position above the cranium, and includes a fixing piece 51 and a division groove 52 as illustrated in FIGS. 3 and 4.

The fixing piece 51 is a fixing member which is installed inside the case part in one direction to fix and support the magnetic field generation part 30 at a predetermined position above the cranium, and as illustrated in FIGS. 3 and 6 to 11, the fixing piece 51 includes at least one of a first fixing piece 51a, a second fixing piece 51b, or a third fixing piece 51c, and variously changes a disposed state of the magnetic field generation part 30.

The first fixing piece 51a is a fixing member which is installed on a central area of the lower case part 10 and fixes and supports the magnetic field generation part 30 at a central area of the cranium, and the first fixing piece 51a may position the magnetic field generation part 30 at the central area of the cranium or the scalp to perform the magnetic stimulation treatment.

The second fixing piece 51b is a fixing member which is installed on a front area of the lower case part 10 and fixes and supports the magnetic field generation part 30 at a front area of the cranium, and the second fixing piece 51b may position the magnetic field generation part 30 at the front area of the cranium or the scalp to perform the magnetic stimulation treatment.

The third fixing piece 51c is a fixing member which is installed on a rear area of the lower case part 10 and fixes and supports the magnetic field generation part 30 at a rear area of the cranium, and the third fixing piece 51c may position the magnetic field generation part 30 at the rear area of the cranium or the scalp to perform the magnetic stimulation treatment.

The division groove 52 is a separation fixing member divided in an upper end of the fixing piece 51, and the division groove 52 is coupled into a support hole defined in the support piece 44 of the spacer 40 and is separated to be spaced outward so that coupling and attaching/detaching between the fixing part 50 and the spacer 40 are easily achieved.

The cooling part 60 is a cooling member which is installed in the inner space of the case part and blows air to the magnetic field generation part 30 so as to cool the magnetic field generation part 30, and the cooling part 60 includes a cooling fan 61 and a cooling hole 62.

The cooling fan 61 is a cooling member which is installed on an area of the case part in one direction and blows the air to the magnetic field generation part 30 to cool the magnetic field generation part 30, and as illustrated in FIG. 12, the cooling fan 61 includes at least one of a first cooling fan 61a and a second cooling fan 61b.

The first cooling fan 61a is a cooling member which is installed an one area of the case part in the one direction and blows the air to the magnetic field generation part 30 to cool the magnetic field generation part 30, and the first cooling fan 61a is installed on a front area of the case part and blows the air inward from the front area of the case part to cool the heat generation of the magnetic field generation part 30.

The second cooling fan 61b is a cooling member which is installed an one area of the case part in the other direction and blows air to the magnetic field generation part 30 to cool the magnetic field generation part 30, and the second cooling fan 61b is installed on a rear area or an upper area of the case part and blows the air inward from the rear area or the upper area of the case part to cool the heat generation of the magnetic field generation part 30.

The cooling hole 62 is a cooling member which passes through the one area of the case part in the one direction and is opened so as to allow outside air to be introduced into the inner space of the case part or to discharge the air from the inside, and the cooling hole 62 includes at least one of a first cooling hole or a second cooling hole.

The first cooling hole is a cooling hole passing through the front area of the case part, and allows the outside air to be introduced inward from the front area of the case part or to discharge the air from the inside to the front area so that the heat generation of the magnetic field generation part 30 is cooled.

The second cooling hole is a cooling hole passing through the rear area or the upper area of the case part, and allows the outside air to be introduced inward from the rear area or the upper area of the case part or to discharge the air from the inside to the rear area or the upper area so that the heat generation of the magnetic field generation part 30 is cooled.

The present invention has been described but may be embodied in different forms without departing from the technical spirit or the main features of the present invention. Thus, the embodiments are disclosed only for illustrative purposes and should not be construed as limiting the present invention.

1. A helmet-type magnetic stimulation device for providing non-invasive treatment by applying a magnetic field to cranial nerve cells or scalp cells, the helmet-type magnetic stimulation device comprising:
a helmet-type case part;
a magnetic field generation part installed in an inner space of the case part and configured to generate the magnetic field; and
a fixing part installed inside the case part in one direction and configured to fix and support the magnetic field generation part at a predetermined position above a cranium.

2. The helmet-type magnetic stimulation device of claim 1, wherein the case part comprises:
a lower case part mounted to be spaced a predetermined distance upward from the cranium; and
an upper case part coupled to an upper portion of the lower case part to define an inner space.

3. The helmet-type magnetic stimulation device of claim 2, wherein the lower case part comprises:
a lower case mounted to be spaced a predetermined distance upward from the cranium;
a cooling guide provided upright on an upper surface of the lower case in a longitudinal direction;
a coupling piece which protrudes from an outer circumference of the lower case and to which the upper case part is coupled; and
a spacing piece which is provided in plurality to protrude from a lower surface of the lower case and is brought into contact with a scalp surface.

4. The helmet-type magnetic stimulation device of claim 1, wherein the magnetic field generation part comprises wound coils in a plurality of layers spaced a predetermined winding distance from each other.

5. The helmet-type magnetic stimulation device of claim 4, further comprising a spacer configured to support lower portions of the wound coils so as to maintain the winding distance of the wound coils to be constant.

6. The helmet-type magnetic stimulation device of claim 5, wherein the spacer comprises:
a spacing plate installed in the inner space of the case part;
a first spacing groove recessed in each of both ends of an upper surface of the spacing plate in one direction;
a second spacing groove recessed in each of both ends of the upper surface of the spacing plate in the other direction; and
a support piece extending outward from an outer circumference of the spacing plate.

7. The helmet-type magnetic stimulation device of claim 1, wherein the fixing part comprises at least one of:
a first fixing piece installed on a central area of the case part and configured to fix and support the magnetic field generation part at a central area of the cranium;
a second fixing piece installed on a front area of the case part and configured to fix and support the magnetic field generation part at a front area of the cranium; or
a third fixing piece installed on a rear area of the case part and configured to fix and support the magnetic field generation part at a rear area of the cranium.
